(19) United States
(12) Patent Application Publication
(10) Pub. No.: US 2014/0349316 A1
(43) Pub. Date: Nov. 27, 2014

(54) USE OF TUMOR NECROSIS FACTOR (TNF) RECEPTOR-ASSOCIATED FACTOR 7 (TRAF7) AS BIOMARKER FOR SYSTEMIC LUPUS ERYTHEMATOSUS (SLE), METHOD AND KIT FOR DETECTING OF HUMAN WITH SLE

(71) Applicant: National Central University, Taoyuan (TW)

(72) Inventors: Shir-Ly Huang, Taipei City (TW); Shin-Chang Lin, Taipei City (TW); Wei-Hsin Sun, Taipei City (TW)

(73) Assignee: National Central University, Taoyuan (TW)

(21) Appl. No.: 14/091,325

(22) Filed: Nov. 26, 2013

(30) Foreign Application Priority Data
May 23, 2013 (TW) 102118255

(51) Int. Cl. G01N 33/543

(52) U.S. Cl. CPC G01N 33/54306
USPC 435/7.92

(57) ABSTRACT

A use of tumor necrosis factor (TNF) receptor-associated factor 7 (TRAF7) as a biomarker for systemic lupus erythematosus (SLE) and a method and a kit for detecting human with SLE are provided. The kit for detecting human with SLE includes TRAF7 and anti-TRAF7 antibodies. The method for detecting human with SLE includes the following steps. A plasma sample is taken from a patient with suspected SLE. The ratio of TRAF7 to total protein in the plasma sample of the patient is compared with that in the plasma sample of normal people. If the ratio of TRAF7 to total protein in the plasma sample of the patient is higher than that of the normal people, then it is possible that the patient is suffering from SLE. TRAF7 can be used as a biomarker for SLE patients in active phase and with renal involvement.

### CROSS-REFERENCE TO RELATED APPLICATION

This application claims the priority benefit of Taiwan application serial no. 102118255, filed on May 23, 2013. The entirety of the above-mentioned patent application is hereby incorporated by reference herein and made a part of this specification.

### BACKGROUND

1. Field of the Application

The invention relates to a protein biomarker, a detection method and a detection kit, and more particularly, to a biomarker for systemic lupus erythematosus (SLE) and a method and a kit for detecting human with SLE.

2. Description of Related Art

Systemic lupus erythematosus is a fairly complex autoimmune disease, and is a quite challenging disease for physicians of rheumatology. The systemic lupus erythematosus, referred to as lupus, is a female predilection systemic autoimmune diseases, and SLE causes an immune response to produce a variety of autoantibodies against omnipresent autoantigens (mostly are nucleic acids and binding proteins thereof in nucleus), wherein dsDNA antibodies are specific for SLE. Clinical symptoms of the systemic lupus erythematosus are all-inclusive and fickle in nature, and a clinical course thereof is sometimes good and sometimes bad, for instance: rash, photosensitivity, oral ulcers, arthritis, pleurisy, pericarditis, glomerulonephritis, neurological symptoms (cramps), hemolytic anemia, leucopenia, thrombocytopenia and so forth. Current diagnosis for systemic lupus erythematosus is mainly based on the clinical symptoms and abnormality test.

Due to the symptoms of systemic lupus erythematosus being protean, patients in early stage of illness are not easily to be diagnosed, and as a result, possible involvement in organs, such as kidneys, may occur, and in some severe cases, a use of dialysis may be required when the illness is been discovered.

### SUMMARY OF THE APPLICATION

The invention provides a use of tumor necrosis factor receptor-associated factor 7 (TRAF7) as a biomarker for systemic lupus erythematosus, and it is used for detecting whether a patient is suffering from systemic lupus erythematosus.

The invention also provides a method for detecting human with SLE, so as to help in determining whether the human is suffering from systemic lupus erythematosus.

The invention further provides a kit for detecting human with SLE, and it is used for detecting whether the human is suffering from systemic lupus erythematosus.

The tumor necrosis factor receptor-associated factor 7 (TRAF7) of the invention is being used as a biomarker for systemic lupus erythematosus.

The method for detecting human with SLE of the invention includes the following steps. Firstly, a plasma sample is taken from a patient with suspected SLE. Afterward, a ratio of TRAF7 to total protein in the plasma sample of the patient is compared with a ratio of TRAF7 to total protein in the plasma sample of normal people, and if the ratio of TRAF7 to total protein in the plasma sample of the patient is higher than that of the normal people, then it is possible that the patient is suffering from SLE.

In an embodiment of the invention, the ratio of TRAF7 to total protein in the plasma sample of the patient is 1.3 to 1.5 times of that of the normal people.

In an embodiment of the invention, the aforementioned method is used to detect that the systemic lupus erythematosus illness of the patient is in an active phase.

In an embodiment of the invention, the aforementioned method is used to detect that the systemic lupus erythematosus illness of the patient has a possibility of renal involvement.

In an embodiment of the invention, the aforementioned method further includes using a protein quantification method to measure a concentration of TRAF7.

In an embodiment of the invention, the protein quantification method includes an enzyme-linked immunosorbent assay detection method.

The kit for detecting human with SLE of the invention includes a tumor necrosis factor receptor-associated factor 7 (TRAF7) and anti-TRAF7 antibodies.

According to the foregoing, the invention uses TRAF7 as a biomarker for systemic lupus erythematosus, and applies the biomarker to provide the method and the kit for detecting human with SLE. As a result, a possibility of human suffering from systemic lupus erythematosus can be quickly and easily determined, and a disease course of the patient with systemic lupus erythematosus or a possibility of involvements in specific organs can be understood, and thereby the disease can be controlled and the timing of treatment is avoid from being delayed.

In order to make the aforementioned and other features and advantages of the present application more comprehensible, several embodiments accompanied with figures are described in detail below.

### BRIEF DESCRIPTION OF THE DRAWINGS

The accompanying drawings are included to provide a further understanding of the application, and are incorporated in and constitute a part of this specification. The drawings illustrate embodiments of the application and, together with the description, serve to explain the principles of the application.

FIG. 1 is a schematic flow diagram illustrating a method for detecting human with SLE according to an embodiment of the invention.

FIG. 2 is an electropherogram of total proteins in plasma samples of normal people and SLE patients according to the method for detecting human with SLE in an embodiment of the invention.

FIG. 3 is a schematic flow diagram illustrating another method for detecting human with SLE according to another embodiment of the invention.

FIG. 4 is a bar chart illustrating concentrations of TRAF7 in plasma samples of normal people and SLE patients according to the another method for detecting human with SLE in the embodiment of the invention.

### DETAILED DESCRIPTION OF DISCLOSED EMBODIMENTS

A biomarker for searching systemic lupus erythematosus are mostly investigated in genetic level during the past, whereas the inventor has researched it from a proteomic perspective and, for the first time, discovered a use of tumor necrosis factor (TNF) receptor-associated factor 7 (TRAF7) as a biomarker for systemic lupus erythematosus.

In particular, TRAF7 may be used as the biomarker for systemic lupus erythematosus in active phase with renal involvement. Next, an application of TRAF7 in the method for detecting human with SLE is introduced in the following below.

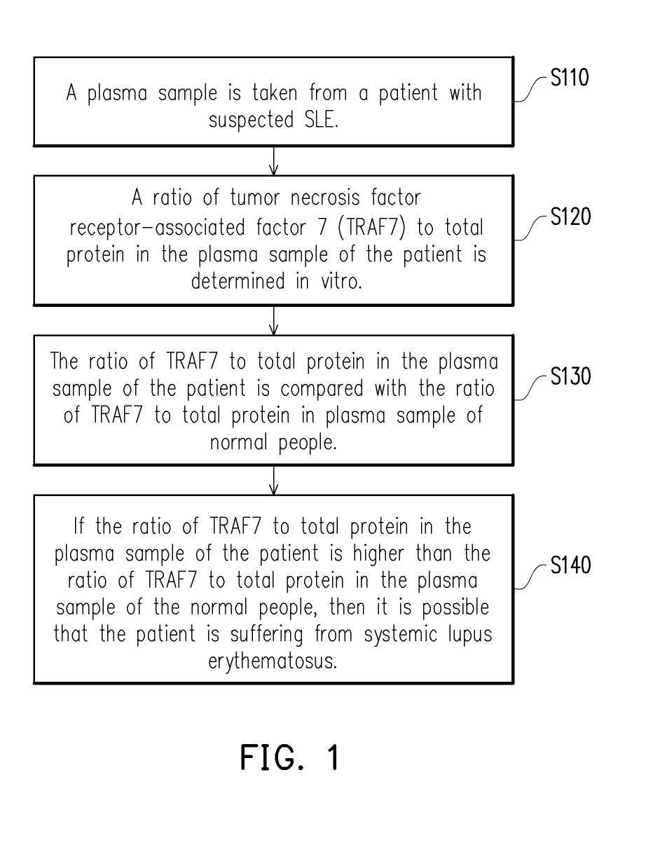

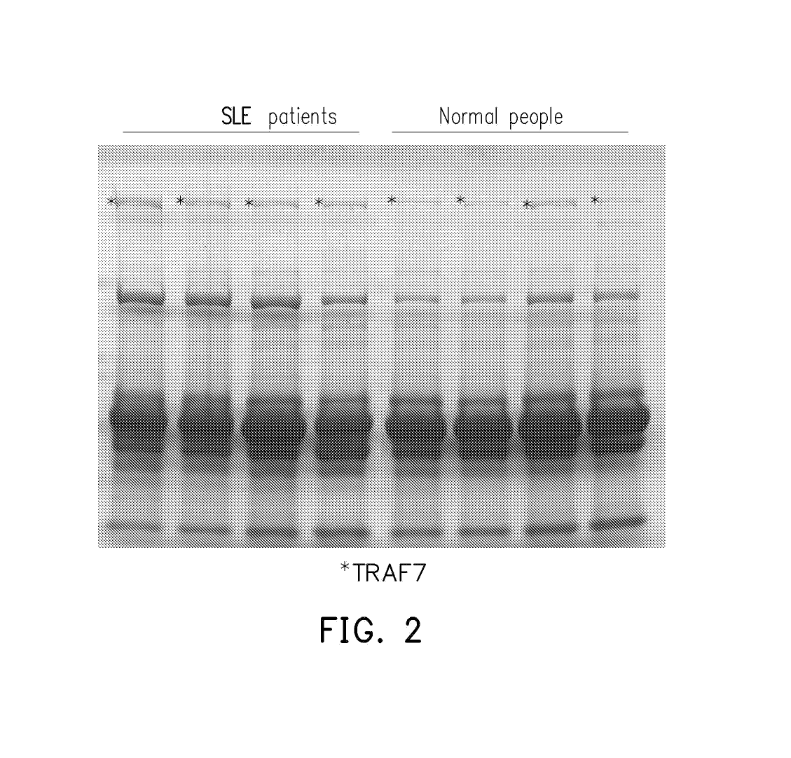

FIG. 1 is a schematic flow diagram illustrating a method for detecting human with SLE according to an embodiment of the invention. FIG. 2 is an electropherogram of total proteins in plasma samples of normal people and SLE patients (clinically diagnosed with renal involvement) according to the method for detecting human with SLE in an embodiment of the invention. Referring to FIG. 1, in the present embodiment, the method for detecting human with SLE includes steps S110-S140. Firstly, the step S110 is performed, such that a plasma sample is taken from a patient with suspected SLE. In the present embodiment, the plasma sample from the SLE patient, for example, is less than 0.1 ml.

Next, the step S120 is performed, such that a ratio of tumor necrosis factor receptor-associated factor 7 (TRAF7) to total protein in the plasma sample of the patient is determined in vitro. In the present embodiment, a method for determining the ratio of TRAF7 to total protein in the plasma sample of the patient and a ratio of TRAF7 to total protein in the plasma sample of normal people in vitro, for example, is to analyze and separate proteins in the plasma samples by a protein electrophoresis and obtain a gel electrophoresis shown in FIG. 2, wherein protein bands of TRAF7 are indicated with [*]. However, a method for detecting the presence of TRAF7 in the plasma may be any protein detection method that can be used for protein identification or quantification, which includes mass spectrometry, enzyme-linked immunosorbent assay (ELISA) and other known protein analysis techniques.

Then, the step S130 is performed, such that the ratio of TRAF7 to total protein in the plasma sample of the patient is compared with the ratio of TRAF7 to total protein in the plasma sample of the normal people. Specifically, “comparing the ratio of TRAF7 to total protein” indicates an amount of relative change of TRAF7 in total plasma protein. It can be known from FIG. 2 that, in the present embodiment and in the same total plasma proteins of the patient with suspected SLE and the normal people, the amount of TRAF7 of the patient has a significant increase. In addition, a computer imaging software is further used to analyze concentrations of TRAF7, and in the present embodiment, wherein an average ratio of TRAF7 to total protein in the SLE patients is 2.6% and an average ratio of TRAF7 to total protein in the normal people is 1.9%. Therefore, in the present embodiment, the ratio of TRAF7 to total protein in the plasma sample of the patients is 1.4 time of that in the plasma sample of the normal people. In an embodiment, the ratio of TRAF7 to total protein in the plasma sample of the patient is 1.3 to 1.5 times of that in the plasma sample of the normal people.

Afterward, the step S140 is performed, such that if the ratio of TRAF7 to total protein in the plasma sample of the patient is higher than that of the normal people, then it is possible that the patient is suffering from systemic lupus erythematosus. In an embodiment, when the TRAF7 ratio in the plasma sample of the patient is 1.3 to 1.5 times of that of the normal people, it indicates that the patient may be suffering from systemic lupus erythematosus, and furthermore, it may indicate that the systemic lupus erythematosus illness of the patient is in an active phase.

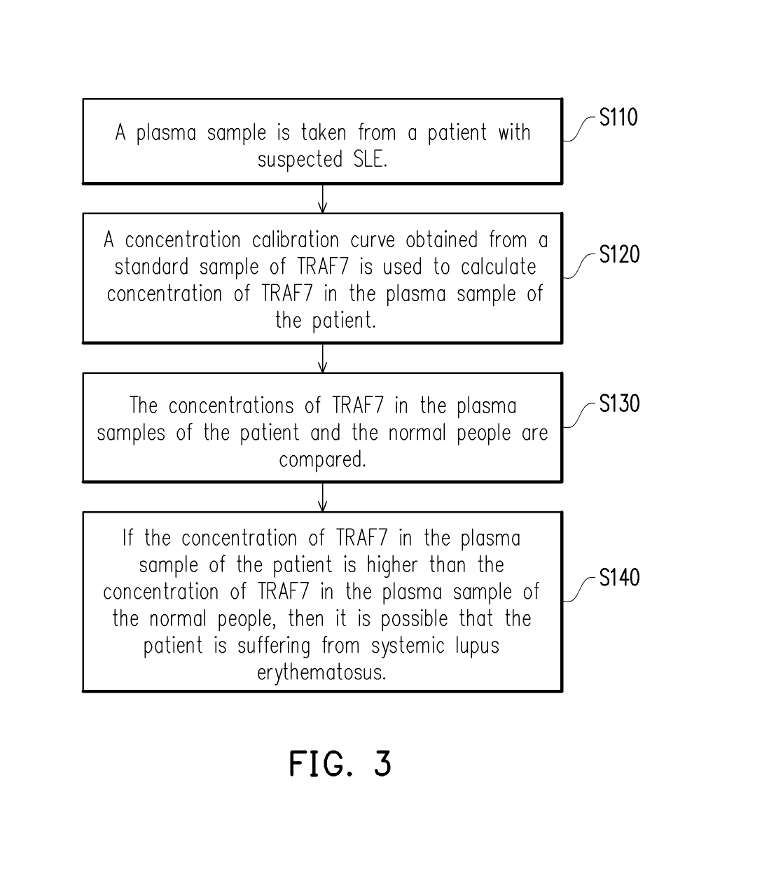

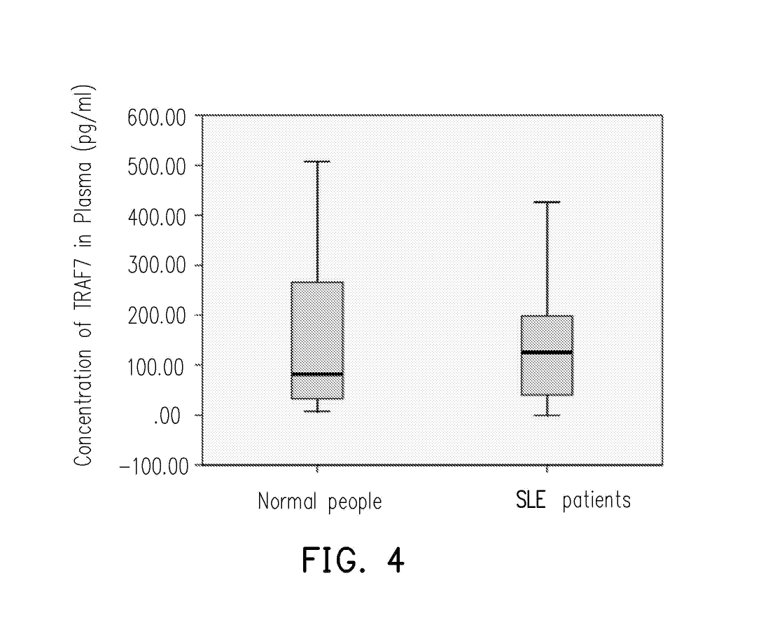

FIG. 3 is a schematic flow diagram illustrating another method for detecting human with SLE according to an embodiment of the invention. FIG. 4 is a bar chart illustrating concentrations of TRAF7 in plasma samples of normal people and SLE patients according to the another method for detecting human with SLE in the embodiment of the invention. A main difference between the procedure described in this embodiment and the previous embodiment is that the concentration of TRAF7 in the plasma is measured. Specifically, as shown in FIG. 3, firstly, the step S110 is performed, such that a plasma sample is taken from the patient with suspected SLE. Next, the step S120 is performed, such that a concentration calibration curve obtained from a standard sample of TRAF7 is used to calculate the concentration of TRAF7 in the plasma sample of the patient. In the present embodiment, a protein quantification method (e.g., an enzyme-linked immunosorbent assay detection method) is used to obtain the concentration calibration curve of the standard sample and calculate the concentration of TRAF7 in the plasma sample of the patient. Then, the step S130 is performed, such that the concentrations of TRAF7 in the plasma sample of the patient and the normal people are compared. In the present embodiment, as shown in FIG. 4, a median of the concentrations of TRAF7 in the plasma samples of 21 patients suspected with SLE, for example, is 125.2 pg/ml (the median is represented with a bold horizontal line), and a median of the concentrations of TRAF7 in the plasma samples of 24 normal people, for example, is 81.5 pg/ml (the median is represented with a bold horizontal line). Therefore, in the present embodiment, a ratio of TRAF7 to total protein in the plasma sample of the patients is 1.5 times of that in the plasma samples of the normal people. Afterward, the step S140 is performed, such that if the concentration of TRAF7 in the plasma sample of the patient is higher than the concentration of TRAF7 in the plasma sample of the normal people, then it is possible that the patient is suffering from systemic lupus erythematosus.

In addition, the inventor provides a kit for detecting human with SLE that includes a tumor necrosis factor receptor-associated factor 7 (TRAF7) and anti-TRAF7 antibodies. Namely, this kit detects the presence of TRAF7 and the ratio of tumor necrosis factor receptor-associated factor 7 (TRAF7) to total protein or the concentration of TRAF7 through using the anti-TRAF7 antibodies, so as to determine whether the human is suffering from systemic lupus erythematosus. The kit for detecting human with SLE may be any kit capable of detecting TRAF7, such as an instrument for mass spectrometer, a kit for ELISA and other known kit for protein analysis, which include TRAF7 and the anti-TRAF7 antibodies.

In the aforementioned embodiment, TRAF7 is being used as the biomarker for systemic lupus erythematosus, and this biomarker is applied to provide the method and the kit for detecting human with SLE. As compared to the conventional biomarkers for systemic lupus erythematosus that are mostly investigated in genetic level, the aforementioned embodiment uses TRAF7 protein as the biomarker for systemic lupus erythematosus. Since TRAF7 is a known protein, and protein detection techniques such as mass spectrometer and ELISA have been developed maturely, a plurality of methods and kits for detecting human with SLE may be developed. Hence, the method and the kit for detecting human with SLE have advantages of being easy to operate and time saving.

On the other hand, since clinical manifestations of SLE is diverse, patients in early stage of illness are not easily to be diagnosed, and as a result, possible involvement in organs, such as kidney, may occur, and in some severe cases, a use of dialysis may be required when the illness is been discovered. However, the method for detecting human with SLE in the aforementioned embodiment, through obtaining the plasma samples of the patients and detecting ratios of TRAF7 in the plasma samples of the patients and the normal people, can quickly and easily determine the possibility of human suffering from SLE, and understand a disease course of SLE or the possibility of involvements in specific organs. For example, in an embodiment, the patients with SLE in active phase and with renal involvement can be determined through the difference between the ratios of TRAF7 in the plasma samples of the patients and the normal people. Therefore, TRAF7 can be used as biomarker for SLE in active phase and with renal involvement. As a result, it may help physicians in determining patients suffering from SLE and controlling the state of illness for the patients with SLE, and thereby avoid the timing of treatment from being delayed. Hence, the biomarker for SLE, the method and the kit for detecting human with SLE can be broadly applied in academic research and clinical diagnosis, and provide great helps in SLE research progress and in controlling the state of illness for SLE patients.

In summary, in the invention, TRAF7 is used as the protein biomarker for SLE, and this biomarker is applied to provide the method and the kit for detecting human with SLE. As compared to the conventional biomarkers for SLE that are mostly investigated in genetic level, the invention uses TRAF7 protein as the biomarker for SLE. Since TRAF7 is the known protein, and the protein detection techniques such as mass spectrometry and ELISA have been developed maturely, many methods and kits for detecting human with SLE may be developed. The method for detecting human with SLE in one embodiment of the invention, through comparing the ratios of TRAF7 in the plasma samples of the patients and the normal people, can quickly and easily determine the possibility of human suffering from SLE, and understand the disease course of SLE or the possibility of involvements in specific organs. As a result, it may help the physicians in determining patients suffering from SLE and controlling the state of illness for the patients with SLE, and thereby avoid the timing of treatment from being delayed. Hence, the biomarker for SLE, the method and the kit for detecting human with SLE can be broadly applied in the academic research and the clinical diagnosis, and provide great helps in the SLE research progress and in controlling the state of illness for SLE patients.

It will be apparent to those skilled in the art that various modifications and variations can be made to the design of the application without departing from the scope or spirit of the application. In view of the foregoing, it is intended that the application cover modifications and variations of this application provided they fall within the scope of the following claims and their equivalents.

1. (canceled)

2. A method for detecting human with SLE comprising the following steps:
obtaining a plasma sample from a patient with suspected SLE;
comparing a ratio of tumor necrosis factor receptor-associated factor 7 (TRAF7) to total protein in the plasma sample of the patient with a ratio of TRAF7 to total protein in the plasma sample of normal people, and if the ratio of TRAF7 to total protein in the plasma sample of the patient is higher than that of the normal people, then it is possible that patient is suffering from systemic lupus erythematosus.

3. The method for detecting human with SLE as recited in claim 2, wherein the ratio of TRAF7 to total protein in the plasma sample of the patient is 1.3 to 1.5 times of that of the normal people.

4. The method for detecting human with SLE as recited in claim 3 being used to detect that the systemic lupus erythematosus illness of the patient is in an active phase.

5. The method for detecting human with SLE as recited in claim 3 being used to detect that the systemic lupus erythematosus illness of the patient has a possibility of renal involvement.

6. The method for detecting human with SLE as recited in claim 2 further comprising using a protein quantification method to measure a concentration of TRAF7.

7. The method for detecting human with SLE as recited in claim 6, wherein the protein quantification method comprises an enzyme-linked immunosorbent assay detection method.

8. A kit for detecting human with SLE, which is used in the method as recited in claim 2, comprising a tumor necrosis factor receptor-associated factor 7 (TRAF7) and anti-TRAF7 antibodies.
